(19) United States
(12) Patent Application Publication
(10) Pub. No.: US 2019/0358630 A1
(43) Pub. Date: Nov. 28, 2019

(54) Microfluidic Devices And Methods For Cellular Thermal Shift Assays

(71) Applicant: The Regents of the University of Colorado, a body corporate, Denver, CO (US)

(72) Inventors: William Old, Boulder, CO (US); Michael H. Stowell, Boulder, CO (US); Yung-Cheng Lee, Boulder, CO (US); Douglas A. Chapnick, Boulder, CO (US); Tristan D. McClure-Begley, Lafayette, CO (US); Xuedong Liu, Niwot, CO (US); Kerri Ball, Longmont, CO (US); Biddut Bhattacharjee, Boulder, CO (US)

(21) Appl. No.: 16/419,054

(22) Filed: May 22, 2019

Related U.S. Application Data
(60) Provisional application No. 62/676,013, filed on May 24, 2018.

(51) Int. Cl. B01L 3/00; G01N 33/50; B01L 7/00; G01N 33/68

(52) U.S. Cl. CPC B01L 3/502715; G01N 33/502; B01L 2300/0627; G01N 33/6803; B01L 2300/1827; B01L 7/52

(57) ABSTRACT

The invention relates to thermal shift assays implemented to uncover ligand protein binding interactions in whole cells or cell extracts. In particular embodiments, the invention provides improved methods and devices for performing thermal shift assays for determining both drug targets and drug mechanism of action. For example, the invention performs thermal shift assays in microfluidic devices and determines ligand-protein binding by relative protein abundance measurements.

### CROSS-REFERENCE TO RELATED APPLICATIONS

The present application claims the benefit of U.S. Provisional patent Application No. 62/676,013 filed on May 24, 2018, which is incorporated herein by reference.

### GOVERNMENT SUPPORT STATEMENT

This invention was made with government support under grant number W911NF-14-2-0019, awarded by the U.S. Army Research Office. The government has certain rights in the invention.

### FIELD OF THE INVENTION

The invention relates to thermal shift assays implemented to uncover ligand protein binding interactions in whole cells or cell extracts. In particular embodiments, the invention provides improved methods and devices for performing thermal shift assays for determining both drug targets and drug mechanism of action. For example, the invention performs thermal shift assays in microfluidic devices and determines ligand-protein binding by relative protein abundance measurements.

### BACKGROUND

Conventional thermal shift assays have been used to determine drug interactions using purified samples and cells by detecting thermodynamic stability changes using a variety of methods including western blot and mass spectrometry.

In a typical thermal shift assay, cellular samples are subjected to a thermal shift by using a thermal block originally designed for use in polymerase chain reaction (PCR) amplification. Multiple samples were required at incrementally increasing temperatures in an effort to derive a protein melting curve. The use of thermal blocks is not ideal as they are not designed for thermal shift assays and variations in the heating time and stability can introduce systematic errors. After thermal block heating exposure, the samples next undergo several steps of lysis and centrifugation which involve multiple sample transfers. These steps are time consuming and also introduce a variety of systematic errors which impair subsequent quantitative analysis. In addition, due to the time restrictions current methods are not capable of performing thermal shift assays at short timescales where early drug-protein interactions occur.

For these and other reasons, current methods are limited with respect to accuracy, efficiency, cost-effectiveness, speed and temporal resolution. Accordingly, there is a need in the art for new methods and devices which provide a different measurement strategy for cellular thermal shift assay.

### SUMMARY OF THE INVENTION

The invention relates to thermal shift assays implemented to uncover ligand protein binding interactions in whole cells or cell extracts. In particular embodiments, the invention provides improved methods and devices for performing thermal shift assays for determining both drug targets and drug mechanism of action. For example, the invention performs thermal shift assays in microfluidic devices and determines ligand-protein binding by relative protein abundance measurements.

In one embodiment, the present invention contemplates a method, comprising: a) providing; i) a microfluidic device comprising at least one microchannel and a plurality of heater elements in contact with said at least one microchannel, and ii) a plurality of cells comprising at least one protein, wherein said at least one protein has a defined melting temperature, iii) a test agent; and b) introducing a first portion of said plurality of cells into a first microchannel; c) introducing second portion of said plurality of cells and said test agent into a second microchannel; c) shifting said temperature in said first and second microchannels to within a defined temperature range with said heating elements; d) measuring a relative protein abundance measurement between said first and second microchannel for said at least one protein; and e) identifying that said protein is bound to said agent by said relative protein abundance measurement. In one embodiment, said relative protein abundance measurement comprises normalized relative protein abundance data. In one embodiment, said shifting of said temperature is performed after a time delay from introducing said second portion of said plurality of cells. In one embodiment, said test agent is a therapeutic agent. In one embodiment, said defined temperature range ranges between 1-3 degrees Celsius of said defined melting temperature of said at least one protein. In one embodiment, said defined temperature range is a defined melting temperature of said at least one protein. In one embodiment, said defined melting temperature ranges between approximately 45-85° C. In one embodiment, said defined melting temperature is 52° C. In one embodiment, said microfluidic device further comprises at least one ultraviolet microchip configured to measure normalize relative protein abundance.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, the term “Tₘ” is used in reference to a protein “melting temperature.” The melting temperature is the temperature at which a population of isolated proteins denature and/or unfold from a quaternary and/or tertiary conformation into a primary and/or secondary conformation.

The term “channels” or “microchannels” as used herein, refer to pathways (whether straight, curved, single, multiple, in a network, etc.) through a medium (e.g., silicon, glass, polymer, etc.) that allow for movement of liquids and gasses. In some embodiments, described herein, “first channel” and “second channel” are used and these need not have the same shape throughout their length. For example, one can change the channel cross-section, curve or split the channel. Channels can connect or be coupled with other components, i.e., keep components “in communication” and more particularly, “in fluidic communication” and still more particularly, “in liquid communication.” Such components include, but are not limited to, liquid-intake ports and liquid outlet ports. “Microchannels” are channels with dimensions less than 1 millimeter and greater than 1 micron. It is not intended that the present invention be limited to only certain microchannel geometries.

The term “microfluidic” as used herein, relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “drug”, “agent” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “test agent” as used herein, refers to any compound or molecule considered a candidate as a peptide binding ligand.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term, “purified” or “isolated”, as used herein, may refer to a peptide composition that has been subjected to treatment (i.e., for example, fractionation) to remove various other components, and which composition substantially retains its expressed biological activity.

Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to “apparent homogeneity” such that there is single protein species (i.e., for example, based upon SDS-PAGE or HPLC analysis). A purified composition is not intended to mean that all trace impurities have been removed.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

As used herein the term “portion” when in reference to a protein (as in “a portion of a given protein”) refers to fragments of that protein. The fragments may range in size from four amino acid residues to the entire amino acid sequence minus one amino acid.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

A “variant” of a protein is defined as an amino acid sequence which differs by one or more amino acids from a polypeptide sequence or any homolog of the polypeptide sequence.

The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

In a related aspect, “microfluidic device” is used to describe a substrate comprising at least one channel and may comprise other defined components including surfaces and points of contact between solutions including, but not limited to, reagents, buffers and/or test compounds.

The term “buffer” as used herein, refers to an aqueous solution consisting of a mixture of a weak acid and its conjugate base, or vice versa. Its pH changes very little when a small or moderate amount of strong acid or base is added to it and thus it is used to prevent changes in the pH of a solution. Buffer solutions are used as a means of keeping pH at a nearly constant value in a wide variety of chemical applications.

The term “buffer mixture” or “buffer system” as used herein, refers to an aqueous solution consisting of a mixture of at least two weak acids and their conjugate base, or vice versa. The mixture pH changes very little over a wide temperature range (e.g., for example, 1-70° C.) due to buffer-buffer interactions that maintain an overall balanced buffer mixture/system pKa. A buffer mixture/system may also contain other components including, but not limited to, salts, ions, metals and/or supplemental buffer molecules.

The term, “Taylor dispersion” as used herein, refers to an effect in fluid mechanics in which a shear flow can increase the effective diffusivity of a fluid. For example, the shear acts to smear out the concentration distribution within a fluid in the direction of the flow, enhancing the rate at which it spreads in that direction.

### BRIEF DESCRIPTION OF THE FIGURES

FIG. 1 presents a schematic of the current state of the art to assess quantitative proteome-wide profiling of protein thermal stability under differential conditions. Savitski et al., (2014). Cells were cultured under differential conditions, such as drug treatment. In an alternative method, the cells were extracted first, and the extracts were treated with drug (“1”). For each condition, the cell or cell-extract sample was divided into 10 aliquots (“2”). Aliquots were subjected to heating at the indicated temperatures. Samples of intact cells were subsequently subjected to extraction with PBS (“3”). After digestion with trypsin, each sample was labeled with a different TMT10 isotope tag (“4”).

FIG. 2A-B presents the liquid chromatography/mass spectrometry data (LC MS/MS) from the state of the art protein-ligand binding melting temperature assay shown in FIG. 1.

FIG. 2A: Exemplary data in the absence of a binding ligand.

FIG. 2B: Exemplary data in the presence of the binding ligand.

FIG. 3 presents a comparative protein-ligand binding melting temperature curve based upon the data in FIG. 2A-B, indicating the melting temperature shift at a 0.5 protein folding/unfolding ratio.

FIG. 4 presents a schematic of the determination of a protein melting temperature shift (Delta X) as measured by a current state in the art method (e.g., Savitski et al. (2014).

FIG. 5A-D presents exemplary data showing four (4) representative protein-ligand interactions identified by a 52° C. MCESTA relative protein abundance (Delta Y; brackets) method that were not identified by a conventional thermal block cycling protein melting point profile shift (Delta X) method.

FIG. 5A: MAPKAPK2.

FIG. 5B: CSNK2A2.

FIG. 5C: PTMS.

FIG. 5D: RABEP2.

FIG. 6 presents an illustrative biological protocol performed by a MCETSA device during a one (1) second interval. For example, proteins with a cell culture (1) is subjected to a treatment (2), denatured by heat (3) and collected for analysis (4).

FIG. 7A-B presents photomicrographs of:

FIG. 7A: A cell culture attached to a microchannel surface.

FIG. 7B: A lysed cell culture on a microchannel surface after digestion/lysis treatment.

FIG. 8 presents one embodiment of an MCETSA system.

FIG. 9 presents one embodiment of a fluid switch comprising at least one switch position.

FIG. 10 In one embodiment, the microfluidic device comprises at least one microchannel (1) comprising a plurality of heaters (12) and sensors (12A). Although it is not necessary to understand the mechanism of an invention, it is believed that each heater and/or sensor is spaced such that the fluid flow passes from one heater and/or sensor to the adjacent heater and/or sensor in approximately 1 second.

FIG. 11A presents one embodiment of an ultraviolet microchip sensor configured to detect relative protein absorbance data. FIG. 11B illustrates that the ultraviolet microchip sensor is also configured to correct for Taylor dispersion anomalies caused by fluid wave front distortions.

FIG. 12A presents one embodiment of a fluidic quick connector.

FIG. 12B presents one embodiment of an MCETSA system configure with a plurality of fluidic quick connectors.

### DETAILED DESCRIPTION OF THE INVENTION

The invention relates to thermal shift assays implemented to uncover ligand protein binding interactions in whole cells or cell extracts. In particular embodiments, the invention provides improved methods and devices for performing thermal shift assays for determining both drug targets and drug mechanism of action. For example, the invention performs thermal shift assays in microfluidic devices and determines ligand-protein binding by relative protein abundance measurements.

### I. Conventional Cellular Thermal Shift Assays (CETSA)

Comparative melting temperatures (Tₘ) for evaluating drug binding, e.g. protein kinase drug targets, to protein targets in cancer cell lines using a CETSA method combined with quantitative mass spectrometry have been reported. Cells, or cellular extracts, were treated with one of several drugs including staurosporine. Fold-changes were calculated by using the lowest temperature condition as the reference. Protein fold-changes were normalized. Normalization of data included ‘fraction non-denatured’ (folded/insoluble protein) for identifying a difference in a calculated Tₘ across the temperature range scan as a change in X (i.e. Delta X). There was no description of normalized protein abundance data determined at or close to a defined protein melting temperature, or use of a microfluidic device. This reference does not describe the use of a microfluidic device to determine relative protein abundance measurement at or within a protein melting temperature to assess ligand protein binding. Savitski, et al. “Tracking cancer drugs in living cells by thermal profiling of the proteome.” Science 346(6205):55 (Summary) and 1255784-1-1255784-10 (Research Article) (2014).

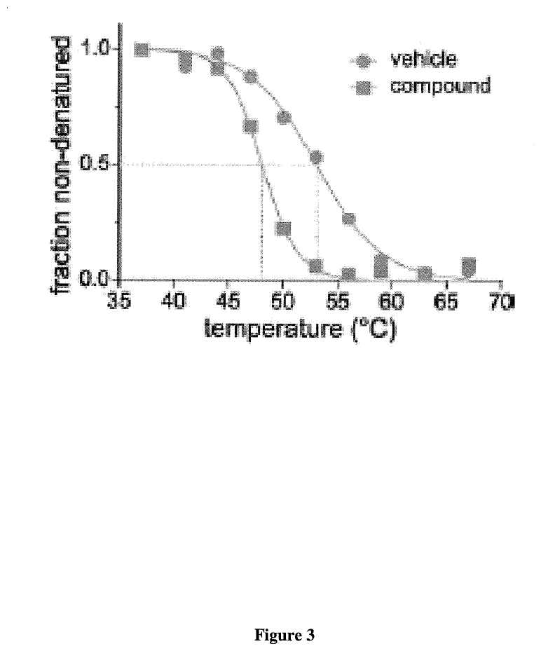

Human non-small cell lung cancer cells have been used in a cellular thermal shift assay (CETSA) combined with analysis by mass-spectrometry of digested proteins' peptides labeled with 10plex Tandem Mass Tags (TMT) for discovering that a drug, i.e. brusatol, regulates Nrf2 activity. More specifically, the melting point of cellular proteins (melting temperature (Tₘ)) was determined by calculating the temperature from the fitted curve at 50% protein abundance. In fact, FIG. 3(C) shows MS abundance data for cystatin C, IGFBP4, and PAF over the temperature ranges used: X axis—temperature points to heat intact cells in the CETSA assay. Y axis-normalized protein abundance (relative intensity) at each temperature point normalized to the average of DMSO and brusatol-treated protein abundance. This reference does not describe the use of a microfluidic device to determine relative protein abundance measurement at or within a protein melting temperature to assess ligand protein binding. Vartanian, et al., “Application of Mass Spectrometry Profiling to Establish Brusatol as an Inhibitor of Global Protein Synthesis.” Mol Cell Proteomics 15(4):1220-12231 (2016).

A version of a cellular thermal shift assay using a label-free method for proteome (whole cell) drug target identification combined with in-gel fluorescence difference caused by thermal stability shift (S-FITGE) combined with mass spectrometry has been reported. Western blots were used to confirm identify of the MS identified protein. In contrast with other methods (e.g., quantitative MS-based thermal proteome profiling (TPP) measuring a melting curve shift along the x-axis), this assay revealed a longitudinal Tₘ comparison based upon deviation along the y-axis between two melting curves. Thermally shifted spots in TS-FITGE 2D electrophoresis of cellular thermal shifted proteins, with and without a drug, were heated and then labeled with Cy2, Cy3, and Cy5 which were considered to be potential target proteins and excised for identification by mass spectrometry. This reference does not describe the use of a microfluidic device to determine relative protein abundance measurement at or within a protein melting temperature to assess ligand protein binding. Park, et al. “Label-free target identification using in-gel fluorescence difference via thermal stability shift” Chem. Sci., 8:1127-1133 (2017).

Cellular thermal shift assay (CETSA) drug target engagement in human cancer cell lines, e.g. K562, mouse cancer cells, mouse livers and mouse kidneys western blot analysis have been described. Multiple aliquots of cell lysate were heated to 9-10 different temperatures with a thermal cycler, between 40° C. and 90° C. After cooling, the samples were centrifuged to separate soluble fractions from precipitated proteins. The presence of the target protein was quantified in the soluble fraction by Western blotting. Relative band intensity (normalized) was graphed against temperature to obtain melting curves, relative to controls, for determining drug target engagement. This reference does not describe the use of a microfluidic device to determine relative protein abundance measurement at or within a protein melting temperature to assess ligand protein binding. Molina, et al., “Monitoring Drug Target Engagement in Cells and Tissues Using the Cellular Thermal Shift Assay” Science 341:84-87 2013.

Cellular thermal shift assays in combination with quantitative mass spectrometry (MS) have been used with denaturing gel electrophoresis and western blot detection using target-specific antibodies, for identifying drug targets the accessible portion of the proteome. Cell lysate, intact cells, biopsies or tissue extracts from a patient, treated or untreated, may provide a sample treated with compounds before heating to at least six temperatures with test compounds added either as a high dose (for melt curve) or, alternatively, as a series of concentrations for a isothermal dose-response fingerprint (ITDRF) CETSA experiment. This reference does not describe the use of a microfluidic device to determine relative protein abundance measurement at or within a protein melting temperature to assess ligand protein binding. Jafari, et al., “The cellular thermal shift assay for evaluating drug target interactions in cells.” Nature Protocols 9(9):2101-2122 (2014).

A cellular thermal shift assay with human cells (K562) heated in a thermocycler and then evaluated by western blotting or mass spectrometry for protein binding to a drug TNP-470 has been described. Differences between the melting curves of corresponding proteins in two patient samples were determined by measuring the differences of soluble protein F for all temperature points. Intensities were normalized and plotted to visualize the changes in melting temperature following ligand treatment. This reference does not describe the use of a microfluidic device to determine relative protein abundance measurement at or within a protein melting temperature to assess ligand protein binding. Norlund et al., “Methods for determining ligand binding to a target protein using a thermal shift assay.” WO 2012/143714.

Thermal shift assay methods have been described for identifying biomarkers (e.g., proteins) indicative of a drug response in a patient by testing treated cells of patients responding to a drug, or before or without a drug treatment, and also compared patients having a reduced response to a drug, analyzed by mass spectrometry. Samples were heated in a PCR thermal block up to a defined temperature for 10 temperatures followed by isobaric labeling for mass spectrometry protein identification. As an example to establish correlations between patient pairs, differences between the melting curves of corresponding proteins in the two patient samples. However, melting curves were graphed comparing relative band intensity (measured from Western blots) to temperature. This reference does not describe the use of a microfluidic device to determine relative protein abundance measurement at or within a protein melting temperature to ligand protein binding. Molina et al., “Method for identifying a biomarker indicative of a reduced drug response using a thermal shift assay” WO 2015/145151.

A microfluidic device with multiple infrared laser heating elements, integrated localized temperature control and integrated optical measurement of fluorescence has been described. The temperature of a single sample can be increased or decreased stepwise using the laser. Fluorescence measurements can be detected for ultraviolet radiation as emitted fluorescence from the samples (especially protein and cells). For example, measurement of the intrinsic fluorescence during thermal excitation enabled the melting temperature (Tₘ) or mid-point of denaturation (Cₘ) of the protein to be measured to determine the thermal stability of a protein, across a range of typically 10-50 different temperatures. Thus the effect of additives, bound ligands or drugs, or protein mutations upon the global protein stability is determined. This reference does not describe the use of a microfluidic device to determine relative protein abundance measurement at or within a protein melting temperature to assess ligand protein binding. Aeppli et al., “Microfluidic Device.” WO 2010/149995.

A microfluidic device has been described having one or more resistive heating elements in thermal contact with the at least one portion of the microfluidic channel and a fluorescence detector for providing a thermal melt analysis (Tₘ) for generating a thermal melting curve using cells. This microfluidic device may be used for receptor-ligand interactions, including those of proteins, for detecting a shift in the thermal denaturation of a protein by a bound molecule. This reference does not describe the use of a microfluidic device to determine relative protein abundance measurement at or within a protein melting temperature to assess ligand protein binding. Sundberg et al., “Method and apparatus for generating thermal melting curves in a microfluidic device.” U.S. Pat. No. 9,376,718 (herein incorporated by reference).

A ligand induced thermal shift assay on a microfluidic device with a heating coil for thermal cycling and a fluorescence detector has been described. The heating coil will typically be disposed adjacent to or in a layer above or below the region of the microchannel wherein heating of the fluid reactants is desired. A shift in the mid-point temperature, the temperature at which the protein is 50% denatured, indicates the protein is bound to a ligand. This reference does not describe the use of a microfluidic device to determine relative protein abundance measurement at or within a protein melting temperature to assess ligand protein binding. Stem S., “Microfluidic devices and methods for performing temperature mediated reactions.” U.S. Pat. No. 6,670,153 (herein incorporated by reference).

Possibilities offered by advances in microfluidic technology for the analysis of therapeutic proteins during manufacturing and formulation have been offered. This reference does not describe the use of a microfluidic device to determine relative protein abundance measurement at or within a protein melting temperature to assess ligand protein binding. Kopp, et al. “Microfluidic approaches for the characterization of therapeutic protein.” J Pharm Sci. Abstract, Jan. 8, 2018.

The Subcellular Pan-Omics for Advanced Rapid Threat Assessment (SPARTA) project has been reported to target the development of a new technological system to rapidly determine how drugs or biological or chemical agents exert their effects on human cells. SPARTA is also developing microfluidic devices to automate sample preparation and control and manipulate individual cell components in order to investigate how protein molecules change in response to a given drug or toxin. As part of the SPARTA project, a microfluidic device was constructed comprising a microchip having dimensions of roughly 3 inches by 1.5 inches. This reference does not describe the use of a microfluidic device to determine relative protein abundance measurement at or within a protein melting temperature to assess ligand protein binding. Anonymous, “National Security: Biomedical innovation in the fast lane.” University of Colorado Boulder Research Report 2014-15.

### II. Microfluidic Cellular Thermal Shift Assays (MCETSA)

In one embodiment, the present invention contemplates microfluidic devices including, but not limited to, integrated thermal control electrodes, flow control systems and/or software. In one embodiment, the microfluidic device is configured to perform a thermal shift assay. In some embodiment, the microfluidic devices are capable of sustaining cell growth, determining drug treatment efficacy, performing cell lysis and sample collection. In one embodiment, the microfluidic devices are used for detecting thermodynamic stability changes within cells. In one embodiment, the microfluidic devices are used for validation of drug targets (e.g., a protein drug target). In one embodiment, the microfluidic devices comprise at least one microfluidic channel, wherein the channel contains a plurality of viable cells adhered to the channel surface. In one embodiment, a test agent is introduced into the channel for treatment of the viable cells. In one embodiment, the test agent includes, but is not limited to, a virus, a drug, an antibody and/or a biologically active compound.

The disclosed microfluidic device and methods (microCETSA: MCETSA) are intended as improvements over conventional devices and methods used for thermal shift assays within cells or cell extracts (CETSA). Specifically, as described herein microfluidic devices having multiple heating elements and ultraviolet-based microchips are intended to replace conventional thermal shift assays using PCR thermal blocks coupled with centrifugation. For example, specific disadvantages of conventional thermal shift assays include thermal block introduction of variations in the heating time and stability that result in systematic errors.

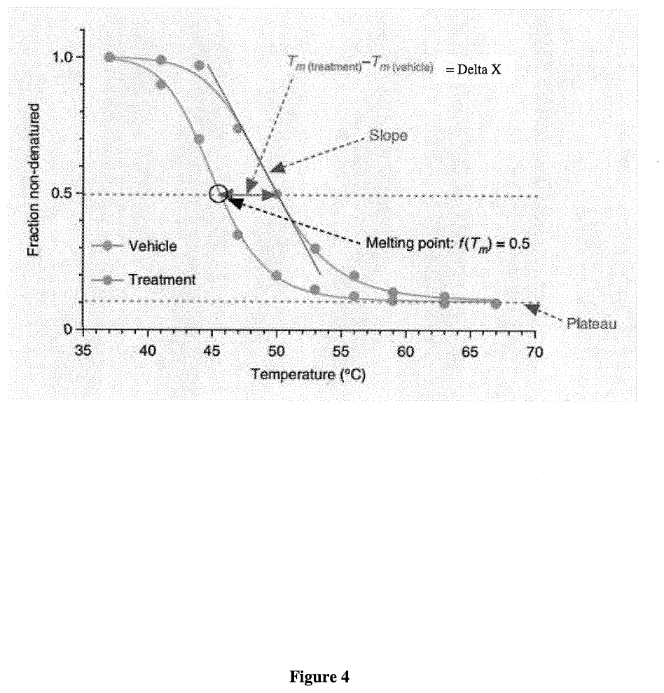

The present invention, as contemplated herein solves the many disadvantages of the current state of the art methods using thermal block cycling to determine whether a protein binds a ligand. For example, one popular method constructs a melting temperature profile for all proteins in a cell extract between a vehicle-treated and a drug-treated condition using a thermal block cycling method. Savitski et al., (2014). In particular, it is the shift in the temperature of melting that is of interest (Delta X=Tₘ(treatment)−Tₘ(vehicle). FIG. 4.

In one embodiment, the present invention contemplates overcoming the above disadvantages by using microfluidic devices. Although it is not necessary to understand the mechanism of an invention, it is believed that one benefit of utilizing a relative protein abundance measurement (e.g., Delta Y), as opposed to Tₘ shifting (Delta X), is that measurements may be taken at a single defined temperature (e.g., a Tₘ) or a small temperature range flanking a Tₘ yields a sufficient statistical difference such that a protein-ligand binding interaction can be identified. In one embodiment, the protein-ligand binding interaction identifies a protein drug target.

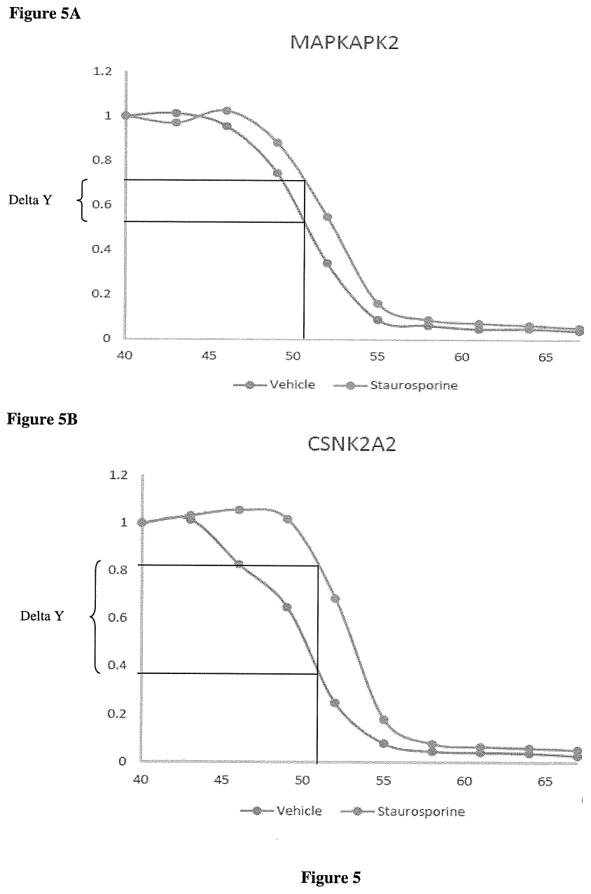

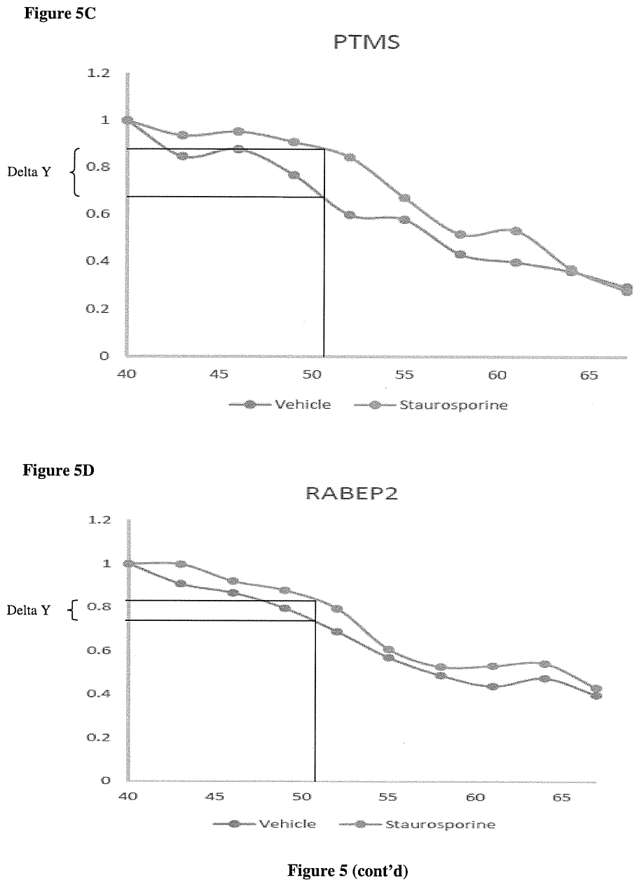

Ligand protein binding interactions determined by the presently contemplated relative normalized protein abundance method was compared to the current state of the art method (e.g., Savitski et al. (2014)) in accordance with Example II. Using 52° C. as a single temperature analysis parameter, fifty-seven (57) known drug-protein interactions were identified using the MCETSA method as described herein. Of these fifty-seven (57) targets, Savitski et al. only identified thirty-one (31). Consequently, the Savitski et al. method failed to identify twenty-six (26) of the fifty-seven (57) known drug targets. For example, four (4) of these twenty-six (6) are MAPKAPK2, CSNK2A1, PTMS and RABEP2. See, FIGS. 5A-5D. These data show that the presently disclosed relative protein abundance measurement (Delta Y) method is unexpectedly superior to conventional methods based upon protein melting temperature shifts (Delta X).

A. Microscale Enhanced Cellular Thermal Shift Assay (MCETSA) Methods

The data presented herein show that comparative results between CETSA and MCETSA methods demonstrate greatly improved testing parameters including, but not limited to, decreased ramp time, increased temperature stability, lower drug treatment times; samples required, i.e. fewer samples, and analysis time, i.e. less time, over conventional cellular shift assays.

MCETSA methods can dramatically extend the limitations of current cellular thermal shift assays. Typical parameters of conventional cellular shift assays were compared between CETSA and MCETSA. MCETSA demonstrated dramatic improvements in cell treatment, thermal shift control, automation and decreased sample handling and sample requirements not only improve data quality but decrease costs. See, Table 1.

TABLE 1
Advantages Of Microfluidic Cellular Thermal Shift Analysis

| | | | Fold |
|---|---|---|---|
| PARAMETER | CETSA | MCETSA | Improvement |
| Ramp Time | 10 seconds | 0.1 second | 100 |
| (37 C. to 70 C.) | | | |
| Temperature | 0.4° C. | 0.1° C. | 4 |
| Stability | | | |
| Drug Treatment | Minutes to Days | Seconds to Days | 10 |
| Times | | | |
| Sample Processing | Manual | Automated | NA |
| Samples Required | 20+ | 4 | 5 |
| Analysis Time | Weeks | Days | 10 |
| Cost (2017 Dollars) | >50,000 | <10,000 | 5 |
| per Drug | | | |
| Sensitivity | TBD | TBD | TBD |
| Specificity | TBD | TBD | TBD |

In one embodiment, the present invention contemplates a method comprising identifying drug binding to a protein. In one embodiment, the protein drug binding is determined by measuring relative protein abundance (Delta Y) at, or close to, a protein's Tₘ. For example, Delta Y may be determined using a thermal shift at a single temperature or a range of temperatures within 1-3 degrees of each other.

In one embodiment, the present invention contemplates that a MCETSA method that is designed not only to achieve improvements over the art but also have characteristics including, but not limited to:
1. Culturing of cells in a microscale device, such that:
   a. Cells have sufficient nutrients.
   b. Cells adhere to a device surface.
   c. Individual cultures of multiple cell types.
   d. Combination cultures of multiple cell types.
2. Treatment of the cells under with an agent, including but not limited to:
   a. Viruses.
   b. Drugs.
   c. Antibodies.
   d. Biologically active compounds.
   e. Any combination of the above.
3. Using a microscale device to:
   a. Contact an agent with a cell population.
   b. Monitor agent contact with a cell population.
   c. Cell population temperature shift to a defined increased temperature.
   d. Cell population temperature shift to a defined decreased temperature.
   e. Lyse and isolate soluble cellular components into a buffer.
   f. Extract insoluble cellular components into a buffer.
4. Quantitatively analyzing a cell proteome for soluble and insoluble cell components using:
   a. Ultraviolet protein absorbance analysis.
   b. Mass spectrometry, liquid chromatography or other suitable methods.
   c. Computational tools including, but not limited to statistical analysis.
5. Assigning statistical significance to insoluble:soluble cell component ratios such that:
   a. A primary mechanism of action of an agent can be identified.
   b. Actionable information can be obtained.
   c. Drug safety can be improved.
   d. Drug metabolite activities can be identified.
   e. Toxin antidotes can be identified.

B. Microscale Enhanced Cellular Thermal Shift Assay (MCETSA) Devices

There are many microscale device configurations that are contemplated by the present invention. Although it is not necessary to understand the mechanism of an invention, it is believed that each of these microscale device configurations can provide the above identified improvements in CETSA. See, Table 1. Some embodiments as contemplated herein comprise characteristics and/or capabilities including but not limited to:
1. An optically transparent substrate with properties including but not limited to:
   a. Culturing between approximately 100K to 1 M cells.
   b. Lithographic deposition of heating elements and temperature sensing elements including, but not limited to:
      i. A 5 nanometer chromium base layer.
      ii. A 100 nanometer gold layer.
      iii. Dimensions of approximately 15×0.3 mm (L×W).
      iv. Between approximately 20 to 30 heating and/or sensor elements spaced approximately 2 mm apart.
   c. A microfluidic chamber bonded to a substrate.
   d. A passivating layer configured on a substrate by atomic layer deposition to prevent electrolysis and/or cavitation.
   e. A substrate having a preferred light wavelength transmission between approximately 250-1100 nm.
   f. A substrate having a preferred composition including, but not limited to, glass, quartz, silicon, and/or plastic.
2. Conductive elements capable of heating and/or temperature sensing with properties including, but not limited to:
   a. Rapid heating from 25° C. to 70° C. within a time ranging between milliseconds to seconds.
   b. Heating and/or sensing elements arranged in a parallel fashion for time dependent activation
3. An atomic layer deposition (ALD) comprised of a combination of 1 or more layers with the compositions, thicknesses and/or properties including, but not limited to:
   a. Titanium dioxide.
   b. Aluminum dioxide.
   c. Alternative passivating materials.
   d. A thickness ranging between approximately 10 nanometers to 200 nanometers.
   e. Heat-induced electrolysis prevention.
4. A microscale (e.g., microfluidic) chamber with properties including, but not limited to:
   a. Bonded to a substrate.
   b. One or more inlet ports.
   c. One or more outlet ports.
   d. A height ranging between approximately 1 to 200 microns.
   e. An anti-turbulence input chamber shape configured to minimize fluid flow distortions (e.g., Taylor dispersions).
   f. A rectangular substrate portion (e.g., 57 mm×23 mm×4 mm; L×W×H) comprising at least one channel (e.g., 50 mm×15 mm×0.2 mm: L×W×H).
5. A photodiode array sensor configured to measure fluid absorbance changes. For example, the sensor may comprise a TSL 1406 Linear sensor (768 pixels; 63.5 μm×55.5 μm) or a TCD 1305DG CCD Linear Image sensor (3648 pixels; 8 μm×64 μm).

In one embodiment, a microfluidic device further comprises an ultraviolet microchip sensor attached to an outflow channel. In one embodiment, the ultraviolent microchip sensor measures a protein concentration variation across a fluidic interface using an ultraviolet absorption spectra. In one embodiment, the ultraviolet microchip provides, real-time, folded:unfolded protein concentration ratio measurements.

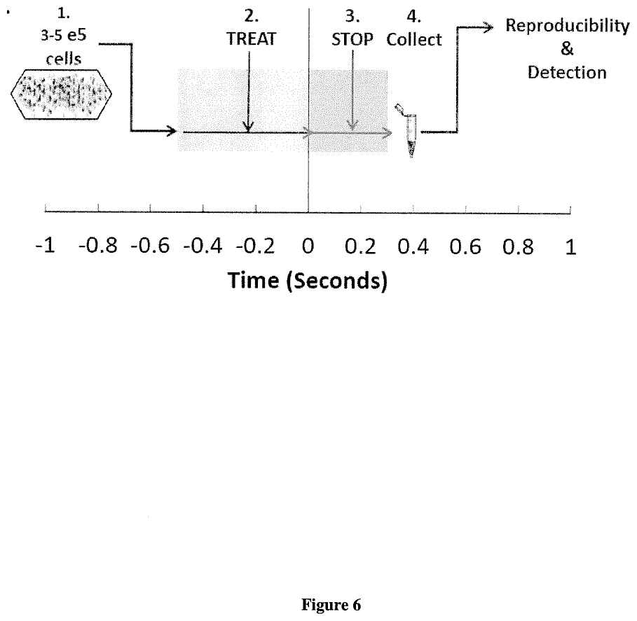

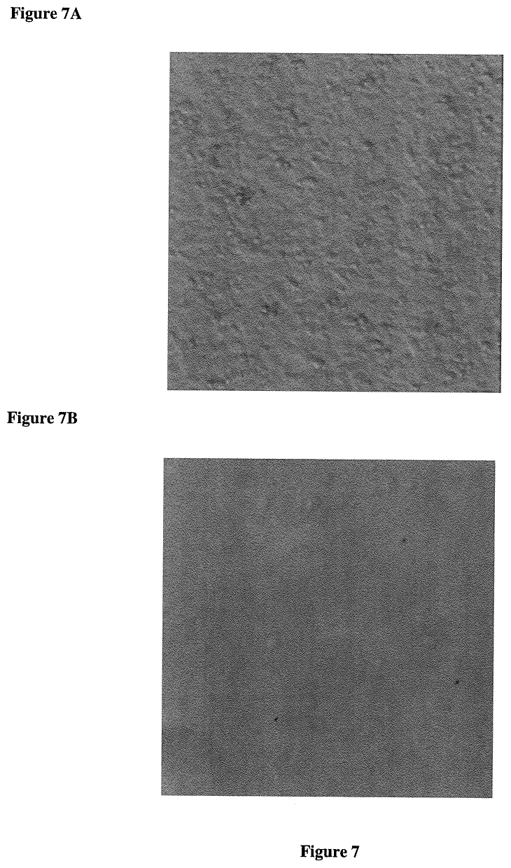

In one embodiment, the present invention contemplates a biologically functional microfluidic device configured to perform a biological protocol. In one embodiment, the biological protocol comprises a microchannel layered with a healthy and viable cell culture. In one embodiment, the cell culture has a cell density of approximately 3.5×10⁵ cells (e.g., for example, spontaneously transformed aneuploid immortal keratinocyte cells; HaCaT cells) within a microchannel. See, FIG. 6, Frame 1 (cell culture); and FIG. 7A. In one embodiment, the microchannel is in fluidic communication with a synchronized and automated fluid switching system. In one embodiment, the fluid switching system provides fluids containing components such as agents, enzymes and/or compounds for the treatment of the cell culture. In one embodiment, these components include but are not limited to, buffers, glucose, minerals, growth factors, hormones and/or digestive enzymes (e.g., for example, pepsin). In one embodiment, the fluid switching system includes, but is not limited to valves, microchannels, chambers and/or an electronic master controller. FIG. 6, Frame 2 (cell treatment). In one embodiment, the fluid switching system delivers a lysis agent (e.g., sodium dodecyl sulphate; SDS). FIG. 7B. In one embodiment, the microfluidic device comprises a plurality of microchannel heaters. In one embodiment, the plurality of microchannel heaters provides heat to denature proteins within the cell culture and/or cell extract. In one embodiment, the plurality of heaters are configured to provide sequential heating to 65° C. within 300 milliseconds. In one embodiment, each of the plurality of heaters are configured within 2 millimeters of a temperature sensor, wherein the temperature sensors are connected the electronic master controller. In one embodiment, the protein heat denaturation step further comprises guanadine, wherein the guanidine reduces protein melting temperatures. In one embodiment, the protein heat denaturation step further comprises tris(2-carboxyethyl) phosphine (TCEP), wherein the TCEP reduces protein disulfide bonds. In one embodiment, the heat denaturation step further comprises a detergent, wherein said detergent dissolves the phospholipid membranes of the cell culture. FIG. 6, Frame 3 (STOP biology). In one embodiment, the biological protocol comprises a step for collecting a sample from the microchannel. In one embodiment, the sample comprises between approximately 75-100 μg of digested protein. In one embodiment, the sample comprises between approximately 50-65 μg of whole cell peptide.

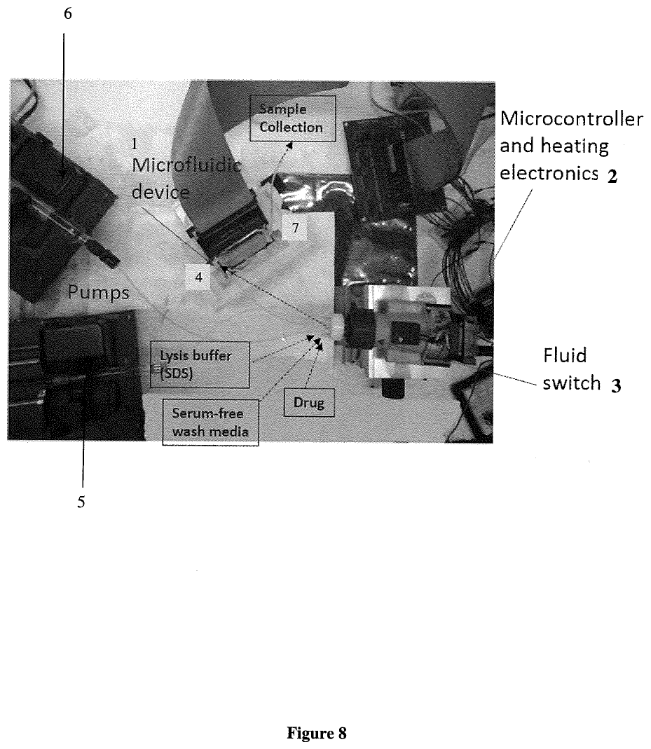

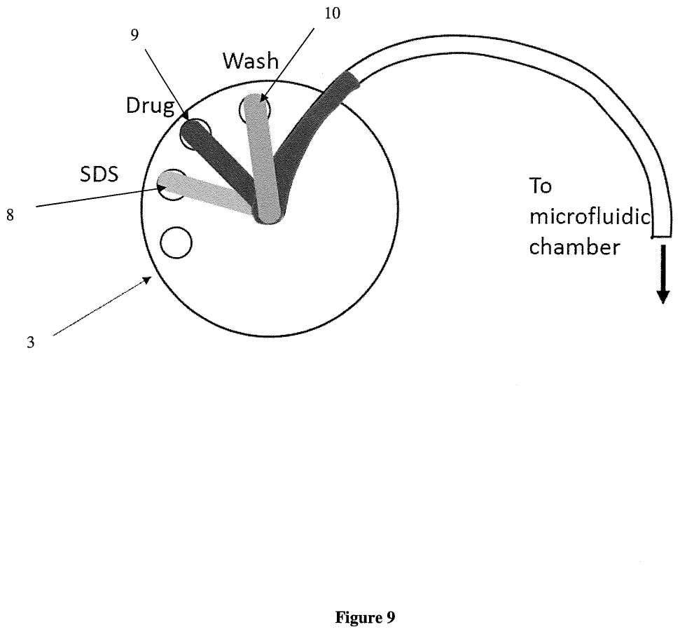

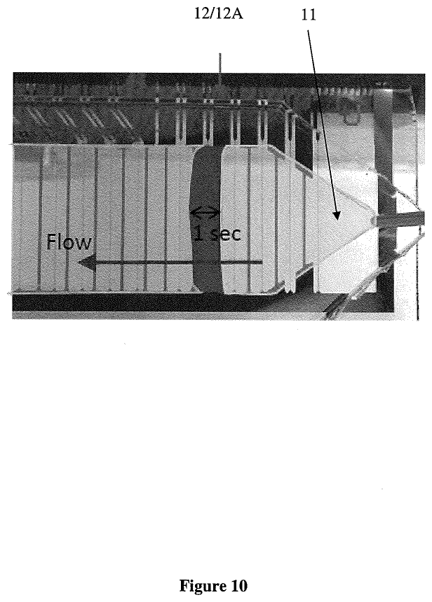

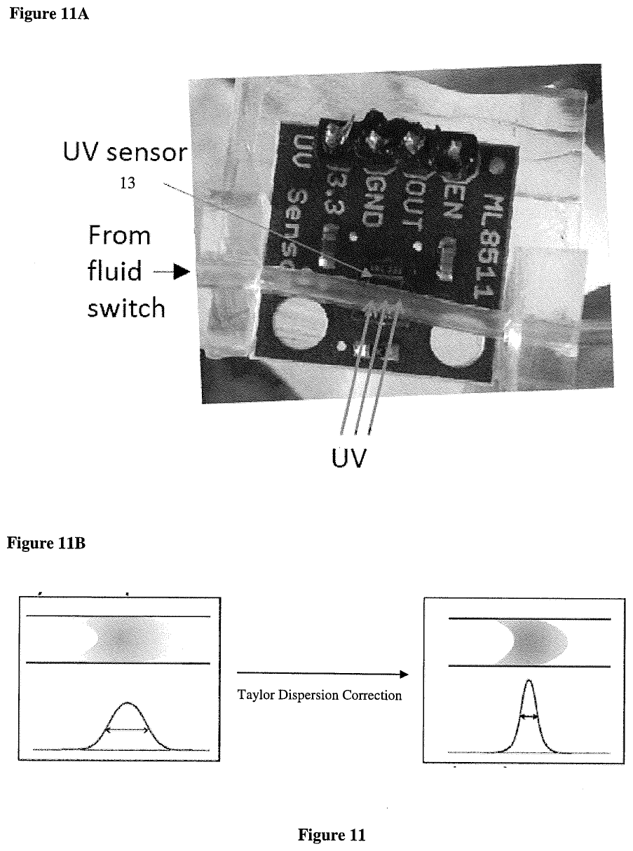

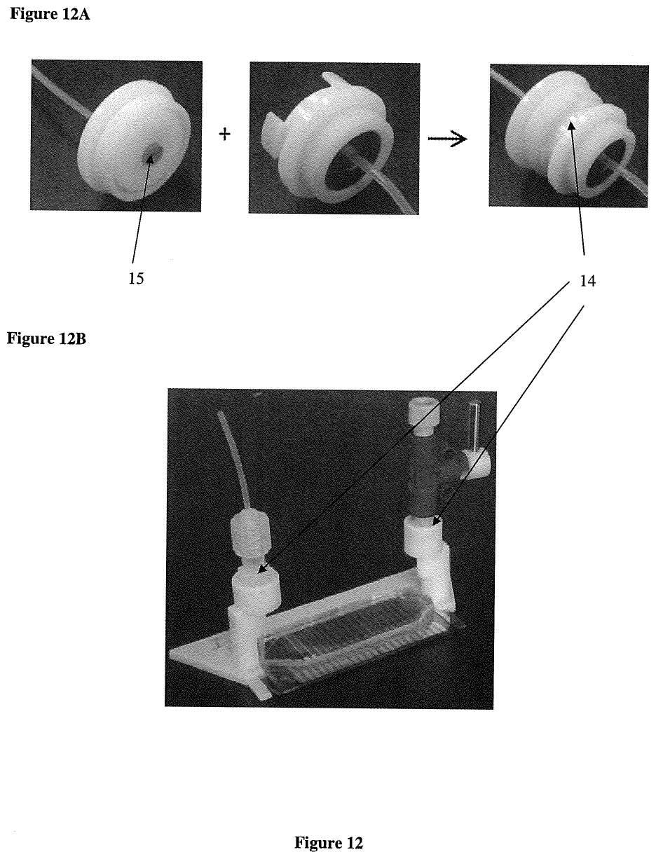

In one embodiment, the present invention contemplates a MCETSA system comprising a microfluidic device (1) attached to an electronics master controller (2), wherein said electronics master controller (2) comprises a fluid switch (3) and a heater electronics board. In one embodiment, the fluid switch (3) is in fluidic communication with an inlet port (4) of the microfluidic device (1). In one embodiment, the fluid switch (3) is in fluidic communication with at least one fluid reservoir (5) containing at least one fluid including, but not limited to, a lysis buffer (e.g., SDS), a serum-free wash buffer (e.g., physiologically buffered saline) and a drug buffer. In one embodiment, the fluid switch (3) is attached to at least one pump (6). In one embodiment, the microfluidic device comprises at least one outlet port (7) for sample collection. FIG. 8. In one embodiment, the fluid switch (3) comprises at least one position. In one embodiment, the at least one position includes, but is not limited to a lysis buffer position (8), a drug buffer position (9) and/or a wash buffer position (10). FIG. 9. In one embodiment, the microfluidic device comprises at least one microchannel (11) comprising a plurality of heaters (12) and sensors (12A). Although it is not necessary to understand the mechanism of an invention, it is believed that each heater and/or sensor is spaced such that the fluid flow passes from one heater and/or sensor to the adjacent heater and/or sensor in approximately 1 second. FIG. 10. In one embodiment, the microfluidic device further comprises an ultraviolet sensor (13) in fluidic communication with said fluidic switch (3). FIG. 11A. Although it is not necessary to understand the mechanism of an invention, it is believed that the ultraviolent sensor (13) comprises an algorithm that corrects protein absorbance data for Taylor dispersion anomalies caused by fluid wave front distortions. FIG. 11B. In one embodiment, the microfluidic device further comprises fluidic quick connectors (14). In one embodiment, the fluidic quick connector comprises a stop-valve (15). FIG. 12A. In one embodiment, a fluidic quick connector is a Luer Lock® connector. In one embodiment, a fluidic quick connector is a magnetic union connector. Although it is not necessary to understand the mechanism of an invention, it is believed that the fluidic quick connectors allow the construction of a microfluidic system that is leak-proof and bubble-free. FIG. 12B.

In one embodiment, the present invention contemplates a method of using an MCETSA system comprising: i) seeding cells in a microchannel of a microfluidic device; ii) culturing said cells in said microchannel; iii) attaching said microfluidic device to said master controller; iv) priming tubes and switches in fluidic communication with said microchannel; v) configuring said fluid switch to wash said cell culture approximately 5-10 times; vi) reconfiguring said fluid switch to release a desired cell lysis solution volume; vii) reconfiguring said fluid switch to release a desired drug solution volume; viii) configuring said electronics board to synchronize heater and/or sensor activation with the tail of a drug solution; and ix) collecting a sample from said outlet port.

C. Microscale Enhanced Cellular Thermal Shift Assay (MCETSA) Device Electronics

In some embodiments, the present invention contemplates microscale device configurations comprising electronic control elements. For example, a MCETSA device as contemplated herein may comprise electronic control elements having characteristics and/or capabilities including, but not limited to:
1. A master controller including, but not limited to:
   a. An Arduino master controller.
   b. An ATmega 1280 (16 MHz) microcontroller.
   c. An ARM Cortex-M4 (MK66FX1M0VMD18, 180 MHz) microcontroller.
   d. Heating power switching components including, but not limited to, a PNP Darlington driver (BD682, 100V 4A 40 W) component or a NPN BJT (BC33725TA, 45V 800 mA 100 MHz 625 mW) component.
   e. A current sensor exemplified by an INA 219 Board (Resolution 0.1-0.8 mA) sensor.
2. A pin interconnect component configured for plug and play connectivity having properties and/or characteristics including but not limited to:
   a. Bonded to a substrate.
   b. One or more input pins.
   c. One or more output pins.

### EXPERIMENTAL

### Example I

### Conventional Determination of Ligand Binding by Melting Temperature Thermal Shifting

This example demonstrates a current state of the art method to determine ligand protein binding by identifying a protein melting temperature shift. Savitski et al., “Tracking Cancer Drugs in Living Cells by Thermal Profiling of the Proteome” Science 346:6205, 1255784-1-1255784-10 (2014).

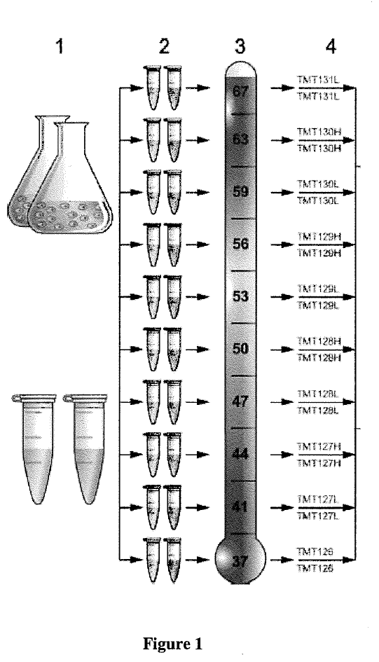

Cells were cultured under differential conditions, such as with and without drug treatment. The cells were extracted first, and the extracts were treated with drug (“1”). For each condition, the cell or cell-extract sample was divided into 10 aliquots (“2”). Aliquots were subjected to heating at the indicated temperatures. Samples of intact cells were subsequently subjected to extraction with PBS (“3”). After digestion with trypsin, each sample was labeled with a different TMT10 isotope tag (“4”). FIG. 1.

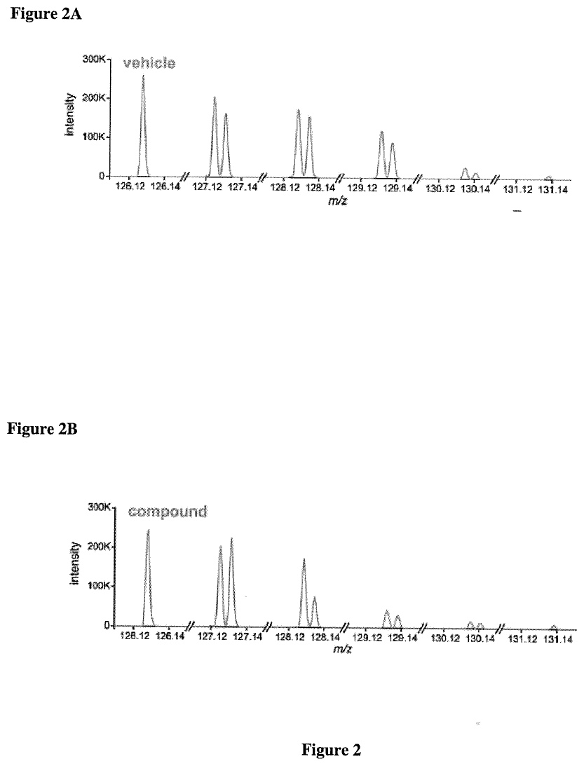

Subsequently, all samples from each condition were mixed (“4”) and analyzed by means of liquid chromatography/mass spectrometry (LC MS/MS (“5”)). FIG. 2A-B.

The obtained reporter ion intensities were used to fit a melting curve and calculate the melting temperature Tₘ of each protein separately for the two conditions (“6”). FIG. 3. The melting temperature shift induced by the ligand protein binding is indicated by the dotted lines. Consequently, this assay identifies ligand protein binding for this particular ligand when the protein melting point is reduced from approximately 54° C. to approximately 48° C.

### Example II

### Improved Relative Protein Abundance (Delta Y) Determination of Protein-Ligand Binding

This example shows the superior performance of a relative protein abundance method (Delta Y) as compared to a protein melting temperature (Delta X) method to identify protein-ligand interactions.

Briefly, a database of defined protein melting point temperatures was constructed. Savitski et al. (2014). These Tₘs were determined by: i) using cell extracts (e.g., K562 cell extracts); ii) mixing the cell extracts with either vehicle or staurosporine for ten (10) minutes. Using a standard PCR heating block, the cell extracts were heated for three (3) minutes followed by cooling for three (3) minutes and then subjected to centrifugation. The soluble fraction (e.g., supernatant) was collected and subjected to in-gel digestion. Each individual protein was then bound to a different TMT10 label. The differentially TMT10 labeled proteins were then subjected to RP/RP LC-MS/MS. This protocol identified the Tₘ for greater than seven thousand proteins (>7,000 proteins).

The Savitski et al. database was used as a source for protein Tₘ information on which to measure relative normalized abundance data using either a single temperature (e.g., at a specific Tₘ) or a selected range of temperatures flanking a Tₘ for a specific protein. The statistical difference between a control measurement and a ligand measurement was calculated using a left-tailed Student's T-test (α=0.01).

The results confirmed that measuring a Delta Y based upon relative normalized protein abundance data successfully identified ligand protein binding interactions when measured by either a single temperature or a small range of selected temperatures flanking a defined temperature (e.g., a protein Tₘ). Improved identification of all ligand protein interactions within a cell extract (e.g., pepsin digested) was found when three (3) different selected temperatures were used in the analysis. Improved identification of all ligand protein interactions within a cell extract (e.g., pepsin digested) was found at 52° C.

We claim:

1. A method, comprising:
a) providing;
i) a microfluidic device comprising at least one microchannel and a plurality of heater elements in contact with said at least one microchannel, and
ii) a plurality of cells comprising at least one protein, wherein said at least one protein has a defined melting temperature,
iii) a test agent; and
b) introducing a first portion of said plurality of cells into a first microchannel;
c) introducing second portion of said plurality of cells and said test agent into a second microchannel;
c) shifting said temperature in said first and second microchannels to within a defined temperature range with said heating elements;
d) measuring a relative protein abundance measurement between said first and second microchannel for said at least one protein; and
e) identifying that said protein is bound to said agent by said relative protein abundance measurement.

2. The method of claim 1, wherein said relative protein abundance measurement comprises normalized relative protein abundance data.

3. The method of claim 1, wherein said test agent is a therapeutic agent.

4. The method of claim 1, wherein said defined temperature range ranges between 1-3 degrees Celsius of said defined melting temperature of said at least one protein.

5. The method of claim 1, wherein said defined temperature range is a defined melting temperature of said at least one protein.

6. The method of claim 1, wherein said defined melting temperature ranges between approximately 45-85° C.

7. The method of claim 6, wherein said defined melting temperature is 52° C.

8. The method of claim 1, wherein said microfluidic device further comprises at least one ultraviolet microchip configured to measure said normalized relative protein abundance.
